(19) United States
(12) Patent Application Publication
(10) Pub. No.: US 2014/0366880 A1
(43) Pub. Date: Dec. 18, 2014

(54) Adjustable non-rebreathing nasal cannula

(71) Applicant: Andrew Donald Metz, Rocky Mount, VA (US)

(72) Inventor: Andrew Donald Metz, Rocky Mount, VA (US)

(73) Assignee: Andrew Donald Metz, Rocky Mount, VA (US)

(21) Appl. No.: 13/920,275

(22) Filed: Jun. 18, 2013

(51) Int. Cl. A61M 16/06; A61M 16/10; A61M 16/12; A61M 16/00; A61M 16/20

(52) U.S. Cl. CPC A61M 16/0672; A61M 16/0078; A61M 16/208; A61M 16/127; A61M 16/1005
USPC 128/204.25; 128/205.11

(57) ABSTRACT

The current embodiment shows a portable, adjustable, oxygen delivery system. This embodiment delivers up to one-hundred percent oxygen while simultaneously conserving oxygen during the user's exhalation phase. The current embodiment contains an air-entrainment system (FIG. 12), a reservoir bag (FIG. 7), a one-way valve assembly (FIG. 5a), and a cannula (FIG. 2a) for delivering a precise amount of oxygen. This embodiment also allows for independent control over oxygen flow and oxygen percentage. Other embodiments are described.

### CROSS-REFERENCE TO RELATED APPLICATIONS

Not applicable.

### FEDERALLY SPONSORED RESEARCH

No federal government funds were used in researching or developing this system.

### SEQUENCE LISTING OR PROGRAM

Not applicable.

### BACKGROUND

### Prior Art

Oxygen delivery is one of the most important requirements of life. The inability of a human to supply oxygen to the body's tissues prevents proper cellular metabolism from occurring. This trend leads to the blood turning acidic, potential loss of consciousness, and a decline in the production of adenosine tri-phosphate (ATP). The ability to produce ATP, necessary for sustaining human life, is not always possible, due to well- known conditions that impair or prevent normal oxygenation. Examples of conditions that affect the absorption of oxygen across the alveolar/capillary membrane (area of the lung where gas exchange occurs) are as follows: chronic obstructive pulmonary disease (COPD), pneumonia, pulmonary fibrosis, or pulmonary edema lead to an increase in oxygen demand. Other conditions such as sickle cell anemia, a patent foramen ovale (PFO), shock, and pulmonary emboli (PE) also lead to increased oxygen demand. Individuals with these conditions, as well as many others, may have an oxygen demand up to five times that of the normal individual just to maintain an oxygen level of ninety percent (the minimum acceptable level to insure proper oxygenation to the body's tissues).

Due to hemoglobin's unique ability to carry oxygen on the red blood cell, little is gained by maintaining oxygen saturation above ninety percent (other than insurance against a drop below that level). Adding much more oxygen is unnecessary, and in some cases will decrease the overall ventilatory effort of some patients, and thus may potentiate respiratory failure.

Current available devices are unable to meet this wide range of oxygen demand and still allow for the patient to be mobile and/or active. The patient is confined to a hospital bed while being attached to high flow oxygen equipment, or placed on mechanical ventilation. When the patient's oxygen requirements exceed fifty to sixty percent Fraction of Inspired Oxygen (FIO2), he/she is required to remain in an acute care setting, as there is no existing device efficient enough to supply the needed percentage of oxygen outside that setting. The development of a portable device will eliminate the necessity of the patient to remain in the acute setting until oxygen requirement sub-threshold has been achieved. Additionally, an aging population will see a growing number of individuals with pulmonary disorders; thereby, increasing the utilization of oxygen devices, hospital admissions, and length of stay. These increases will also result in overburdening an already heavily taxed healthcare system.

To meet the patient's need while decreasing overall healthcare costs there is a need for a device that is efficient enough to deliver higher FIO2 levels with low flow oxygen. This would mean a patient on fifty percent FIO2 or above could be successfully discharged from the acute care setting while still meeting their oxygen need.

Prior U.S. patents developed with oxygen conservation in mind include the following: U.S. Pat. Nos. 7,328,703, 4,054,133, 4,535,767, 4,572,177, 5,666,945, 5,881,725, 6,364,161, 6,612,307, and 6,752,152. In my experience as a healthcare provider, while these devices are aimed at saving oxygen they are unable to fully meet the varied needs of oxygen users. These devices are also unable to provide the high FIO2 levels that are needed by some oxygen users. Other limitations of these devices will now be discussed.

In U.S. Pat. No. 7,328,703 (Tiep, Brian L., Feb. 12, 2008) and U.S. Pat. No. 4,535,767 (Otsap, Ben A., Aug. 20, 1985), air entrainment around the nasal prongs leads to a decrease in the delivered FIO2 of the medical oxygen. In addition, U.S. Pat. No. 7,328,703 is designed to hold approximately 25 milliliters of stored oxygen, however, while the user is inhaling this device allows the user to entrain ambient air into the nasal passages with the oxygen flow, thus decreasing the total inhaled FIO2. For example, during a standard one second inspiration, a patient with this device on six liters of oxygen will only achieve 125 milliliters of pure oxygen while the remainder of the breath will be from ambient air. Given that the average breath taken in by most individuals is five-hundred milliliters, the patient is only inhaling about one fourth of their inspiratory volume from medical oxygen. This would not be tolerable by users with high oxygen demands as this would not allow for a high enough FIO2.

U.S. Pat. No. 4,054,133 (The Bendix Corporation, Oct. 18, 1977), U.S. Pat. No. 6,364,161 (Victor Equipment Company, Apr. 2, 2002), U.S. Pat. No. 6,612,307 (Western/Scott Fetzer Company, Sep. 2, 2003), U.S. Pat. No. 5,666,945 (Salter Labs, Sep. 16, 1997), U.S. Pat. No. 5,881,725 (Victor Equipment Company, Mar. 16, 1999), and U.S. Pat. No. 6,752,152 (Precision Medical, Inc., Jun. 22, 2004), are all pneumatic based systems. These systems are designed to conserve oxygen by preventing delivery of oxygen during the patient's exhalation. However, these systems pose two distinct problems: they can be too heavy for some patients to manage or the patient may be unable to trigger a pneumatic sensor, thereby enabling the inspiratory cycle. Both of the examples above may also increase the anxiety and overall effort for the patient which would, in turn, increase the body's oxygen demand, thus exacerbating the condition of the patient.

In U.S. Pat. No. 4,572,177 (Otsap, Ben A., Feb. 25, 1986), the device poses a risk of infection to the patient due to the requirement of exhaled gases into the system to perform adequately. This may be permissible for patients without active infection; however, the device may become a vector for recurrent infections. This risk may lead to the development of pneumonia and/or sepsis; thus, the device has the potential for serious harm to the patient.

An additional downfall of the above mentioned devices is the inability of each to deliver a precise FIO2. Each device delivers a pre-set liter flow of oxygen, thereby, requiring more oxygen usage to maintain higher FIO2 levels. FIO2 levels may also change, relative to the manner in which the user is breathing; i.e., faster, deeper breathing may decrease FIO2 levels. Fluctuating FIO2 may pose harm to the user if the oxygen level in the blood drops below ninety percent.

Thus, the devices listed above are either unable to meet the patient's need, are too cumbersome for some patients to manage, or there is an increased risk of infection. All of these devices, while aimed at helping oxygen users, fall short of meeting the above listed needs. A remaining need exists for an oxygen system that provides high, precise FIO2 levels with low-flow oxygen that is efficient enough to prevent oxygen waste during exhalation.

### SUMMARY OF THE INVENTION

In order to aid in the understanding of this system, it can be stated in essentially summary form that the current embodiment of this system is to provide three well needed advantages to its users. First, it can provide an exact fraction of inspired oxygen (FIO2) via a nasal route, second, the current system can decrease the liter flow of oxygen required to deliver the same FIO2 in comparison to other systems, and third, the current system can store the oxygen/oxygen mixture while the user is exhaling, thus decreasing the overall oxygen usage of the user.

As normal inspiration in humans is over one second, and exhalation is three seconds or longer, one can see that, with a continuous flow system, a high percentage of oxygen is wasted during exhalation. A user on a standard nasal cannula, with an oxygen flow of six liters a minute, will receive one-hundred milliliters of oxygen, from the system, during inspiration and approximately four-hundred milliliters will be wasted by the system with each breath. To prevent this waste, the current embodiment of the system listed here can collect up to five-hundred milliliters of oxygen if needed. It also allows the user to inhale up to six-hundred milliliters of oxygen with each breath on the same six liters per minute flow, as listed above. Given that an average breath is five-hundred milliliters, the current system listed here can meet the total inspiratory breath of many of its users. In addition, it does not waste the oxygen flow being delivered during exhalation.

As far as FIO2 levels are considered, a normal nasal cannula can only deliver forty-four percent FIO2 with six liters per minute flow, while this current embodiment (as listed above) can provide up to one-hundred percent oxygen with the same liter flow, depending on the level of desired air-entrainment. Other purposes and advantages of this system will become apparent from the following description, as the summary set forth above is inherently incapable of indicating the many purposes, advantages, and/or features that are important to the system.

### BRIEF DESCRIPTION OF THE DRAWINGS

Having thus described the system in general terms, reference will now be made to the accompanying drawings, which are not necessarily drawn to scale, and wherein:

FIG. 1 is a perspective view of the fully assembled current embodiment of the oxygen delivery system.

FIG. 2a is a perspective view of the nasal cannula with the supply tubes attached.

FIG. 2b is an enlarged view of the nasal cannula with the supply tubes broken away.

FIG. 3 is a perspective view of the retaining clip.

FIG. 4 is a perspective view of the cap.

FIG. 5a is a perspective view of one-way valve assembly.

FIG. 5b is a top view of one-way valve assembly.

FIG. 5c is a perspective view of one-way valve assembly on its side.

FIG. 6a is an enlarged top view of the one-way valve.

FIG. 6b is a perspective view of the side of the one-way valve with part of the material elevated.

FIG. 7 is a perspective view of the reservoir bag.

FIG. 8a is a perspective view of first piece of air-entrainment assembly.

FIG. 8b is a rear view of the first piece of air-entrainment assembly.

FIG. 9a is a perspective view of the second piece of air-entrainment assembly.

FIG. 9b is a rear view of the second piece of air-entrainment assembly.

FIG. 10a is a perspective view of safety guard for the air-entrainment system.

FIG. 10b is a rear view of the safety guard for the air-entrainment system.

FIG. 10c is a perspective view of the bottom of the safety guard for the air-entrainment system.

FIG. 11 is a perspective view of the tubular conduit.

FIG. 12 is an exploded view showing the proper assembly of the air-entrainment system.

### REFERENCE NUMERALS

20. Nasal prongs
21. Supply tubes
22. Hole
24. Supply tube Attachment
26. One-way valve
28. Securing unit
30. Retaining unit
31. Base brackets
32. Reservoir bag outlet
34. Reservoir bag inlet
36. Air-entrainment window
38. Guide rim
40. Air-entrainment outlet
42. Oxygen percentage display
44. Oxygen gas outlet
46. Guide flange
48. Oxygen gas inlet
50. Safety guard slot
52. Safety guard hole
54. Safety guard inlet hole
56. Tubing adapter

### DETAILED DESCRIPTION

### First Embodiment

The present system will now be described more fully hereinafter with reference to the accompanying drawings, in which some examples of the embodiments of the system are shown. Indeed, these systems may be embodied in many different forms and should not be construed as limited to the embodiments set forth herein; rather, these embodiments are provided by way of example so that this disclosure will satisfy applicable legal requirements. Like numbers refer to like elements throughout.

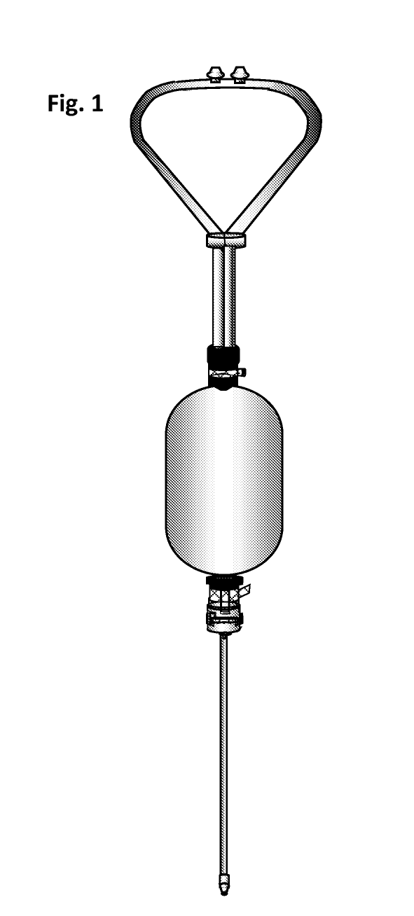

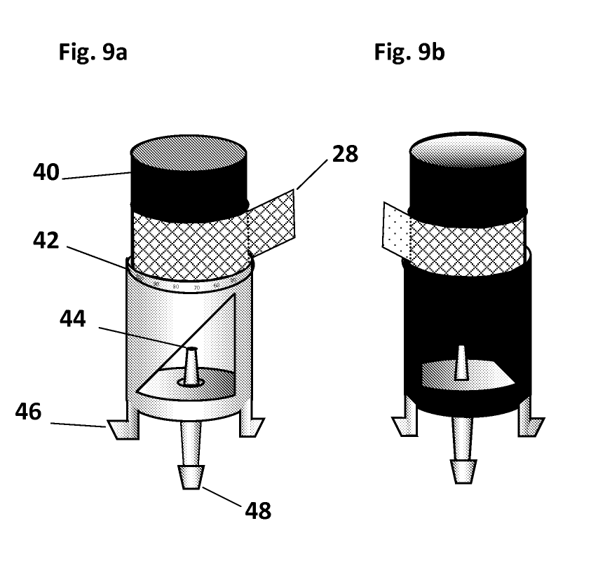

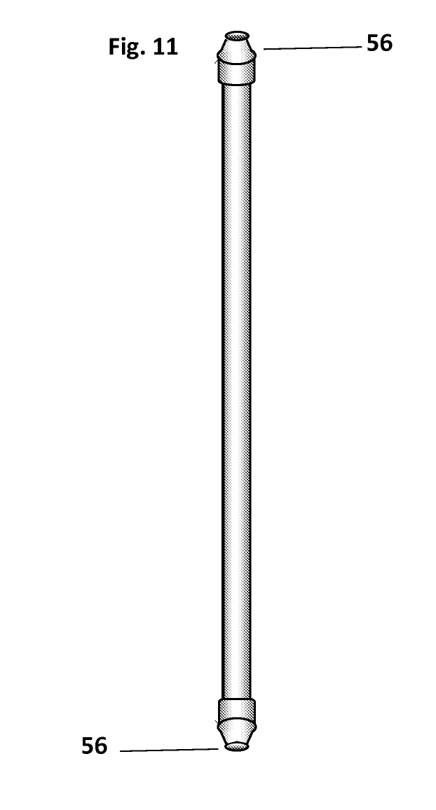

The current embodiment will be described in order of which the flow of gas will enter the system to the point at which it leaves the system. The materials for this embodiment will be made, at least partially, of synthetic and/or natural polymers. The fully assembled perspective view of the current embodiment can be seen in FIG. 1. The first piece of the embodiment to describe is a tubular conduit seen in the perspective view of FIG. 11 which has two tubing adapters 56 attached at adjacent ends. The tubing adapter 56 will attach, at one end, to a standard oxygen supply source, and the adjacent end will attach to an oxygen gas inlet 48 on the second piece of an air-entrainment system seen in FIG. 9a. In addition, an oxygen gas outlet 44 and an oxygen percentage display 42 are also located in FIG. 9a; however, the air-entrainment system must be fully assembled prior to making the above mentioned connection.

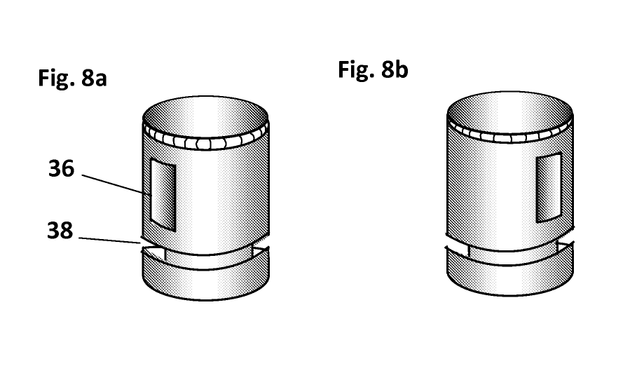

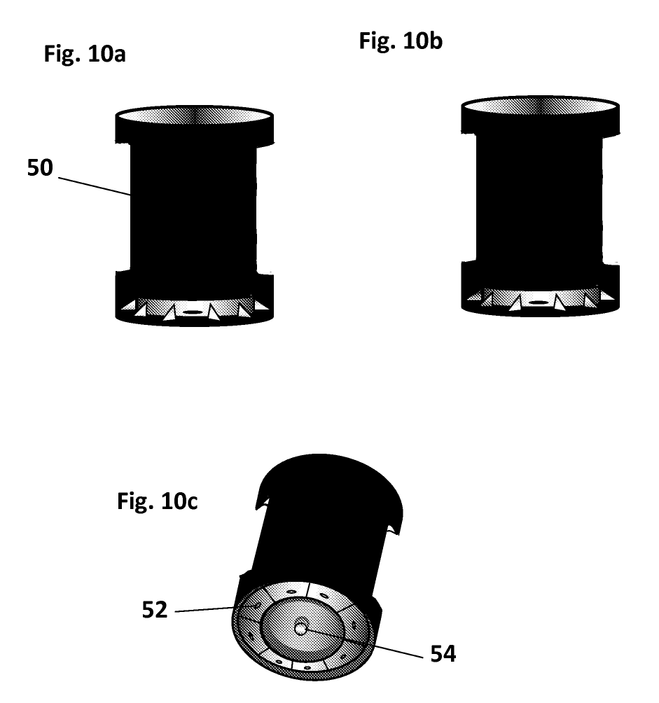

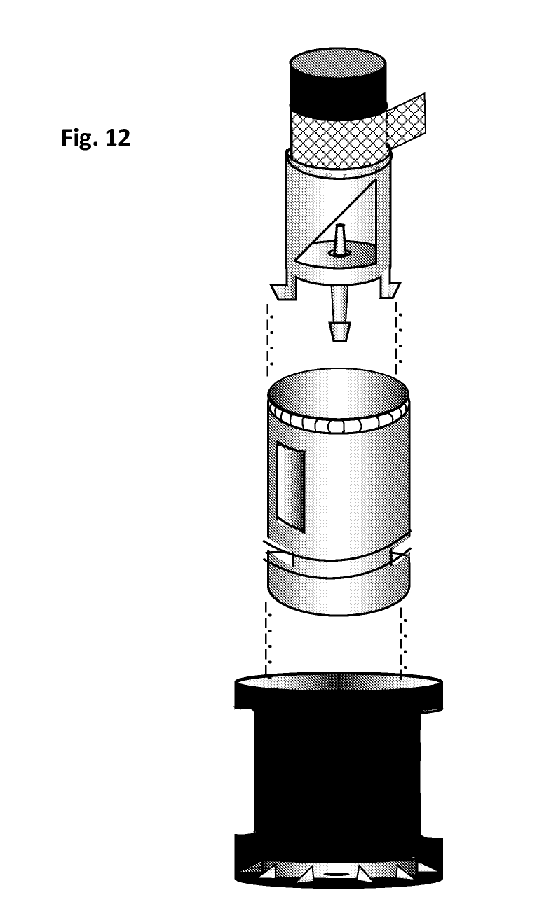

Following is a discussion of the air-entrainment assembly. Proper assembly can be easily seen in the exploded view of FIG. 12. To assemble, take the air-entrainment assembly in FIGS. 9a and 9b, place on top of the air-entrainment system shown in FIGS. 8a and 8b. Press in a downward motion until the guide flange 46, in FIG. 9a, protrudes slightly out of the guide rim 38 in FIG. 8a. FIG. 8a also shows an air entrainment window 36. Next, take the newly assembled air-entrainment system, and place in on top of a safety guard, shown in perspective view, in FIGS. 10a, 10b, and 10c. Press in a downward motion until said inlet 48 (FIG. 9a) passes through safety guard inlet hole 54 shown in FIG. 10c. Safety guard slots 50 are best shown in FIG. 10a, and safety guard holes 52 are best shown in FIG. 10c. Tubing adapter 56 (FIG. 11) can now be pressed onto said oxygen gas inlet 48 (FIG. 9a).

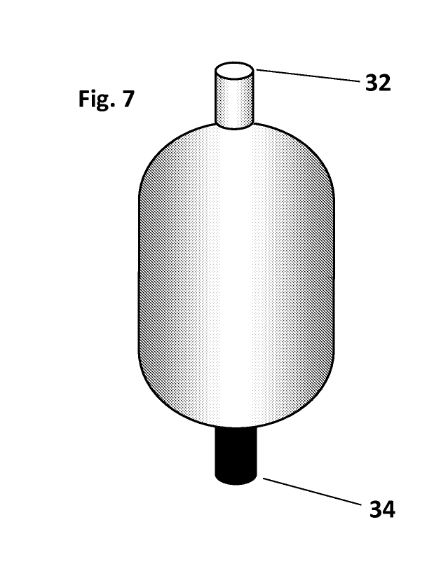

To continue, take air-entrainment outlet 40 in FIG. 9a, and place it into reservoir bag inlet 34 shown in perspective view in FIG. 7. Take securing unit 28, shown in perspective view in FIG. 9a, and place around the reservoir bag inlet, thus securing said air-entrainment outlet 40 to said reservoir bag inlet 48. While this embodiment uses a securing unit similar to that of tape, alternative securing units may be used instead.

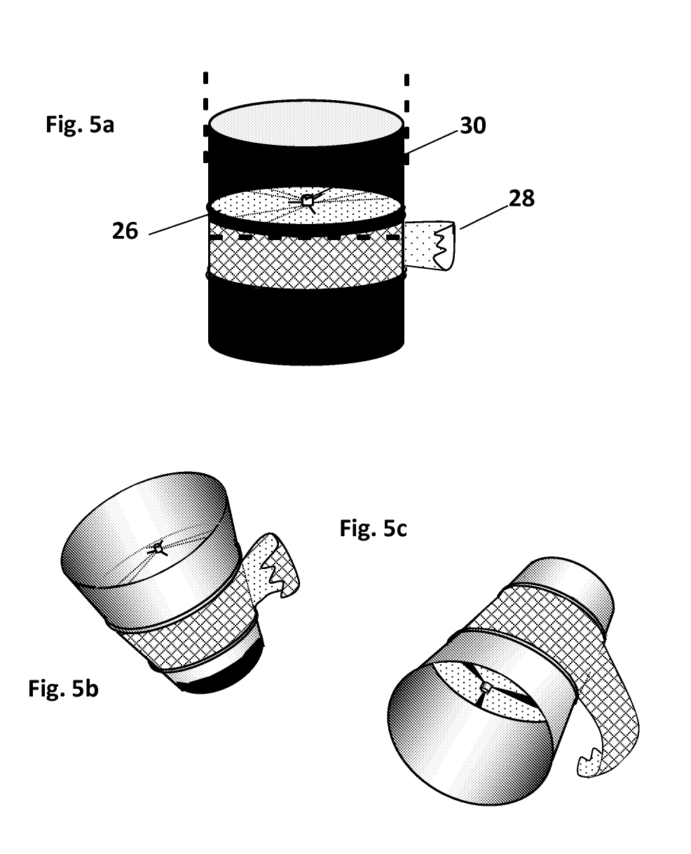

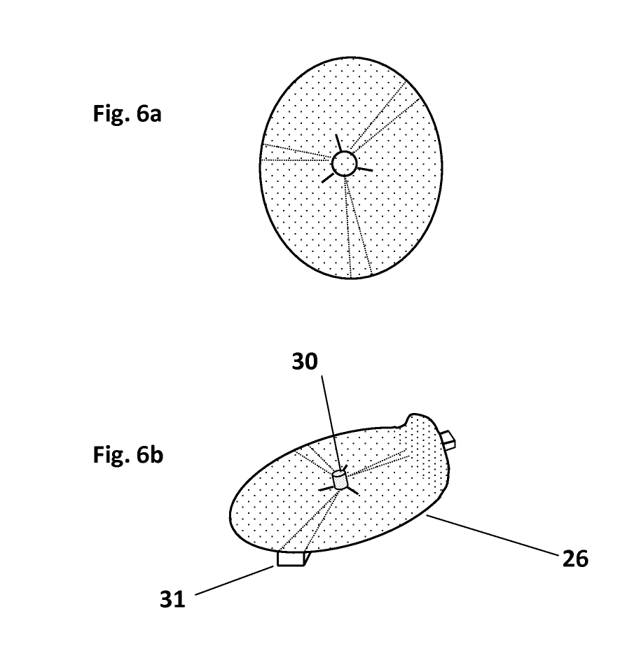

Continuing, take the one-way valve assembly shown in FIGS. 5a, 5b and 5c and place it into reservoir bag outlet 32 shown in FIG. 7. Take securing unit 28, shown in perspective view FIG. 5a, and place around the reservoir bag outlet, thus securing said one-way valve assembly in FIG. 5a to said reservoir bag outlet 32. A one-way valve 26, shown best in exploded view, in FIG. 6a, is made of a material suitable to allow it to bend easily during a user's inspiration. Said one-way valve is placed atop at least three base brackets 31. A securing unit 30 will be placed over said one-way valve 26 attaching to said brackets, all of which can be seen in enlarged view in FIG. 6b.

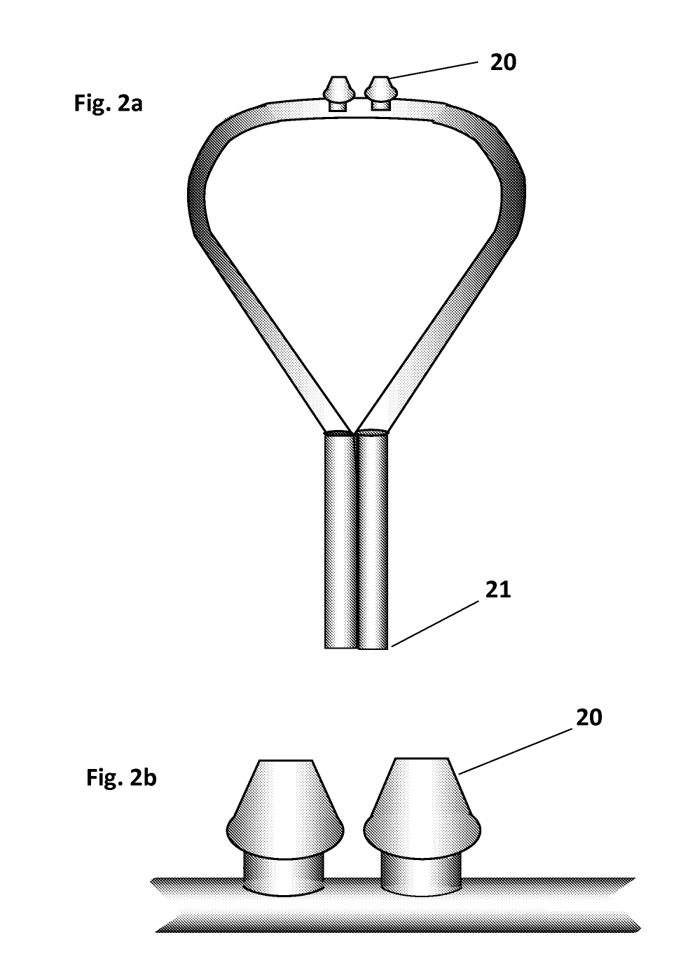

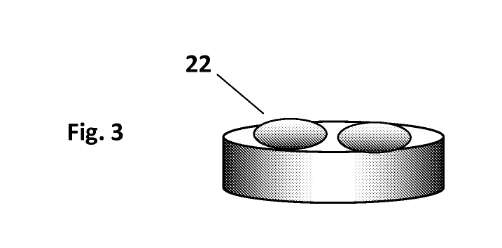

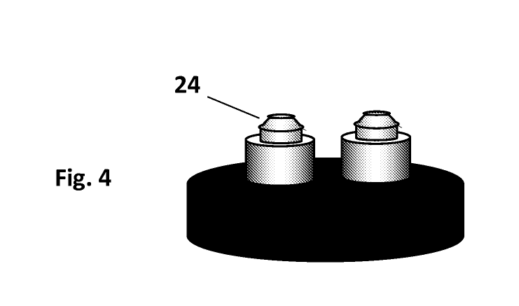

Following this, take cap shown in FIG. 4 and press it, in a downward motion, over said one-way valve assembly shown in FIG. 5a. While not expressly shown, these units would be made to allow them to snap together easily, but not in a manner as to become disconnected as easily. Next, a nasal cannula's supply tubes 21 (FIG. 2a) will be placed through holes 22 in retaining clip, shown in FIG. 3. Now the nasal cannula's supply tubes 21 shown in FIG. 2a will be placed on supply tube attachment 24, shown in FIG. 4. FIG. 2b shows an exploded view of the nasal cannula's nasal prongs 20. This now concludes the full assembly of the current embodiment.

### OPERATION

### First Embodiment

The general purpose of this current embodiment is to provide to an efficient, precise amount of oxygen, up to one-hundred percent, while conserving oxygen during a user's exhalation. A complete assembly of this embodiment can be viewed in FIG. 1. This embodiment is designed to attach to standard oxygen flow systems. It can adjust the percentage of oxygen independent of the flow of oxygen via an air-entrainment system, shown in an exploded view, in FIG. 12. If there is no air entrainment by the system, then one-hundred percent oxygen will be delivered. The system has the ability to store oxygen during a user's exhalation with a reservoir bag, shown in FIG. 7, which is inline. The flow of oxygen need only be set high enough to prevent full bag deflation at the user's end of inspiration, decreasing the overall oxygen usage. The nasal prongs 20 (FIG. 2a) on the nasal cannula also aid in the efficiency of the system by preventing air entrainment into the user's nares during inspiration, thereby preventing a decrease in the oxygen percentage being delivered.

FIG. 11 shows the tubular conduit with both tubing adapters 56 located on adjacent ends. The said adapters allow for the connection of a standard oxygen system to connect to an oxygen gas inlet 44 (FIG. 9a) located on one part of an air-entrainment system. Once said air-entrainment system is assembled, as stated in the previous section, each part serves a unique purpose. FIGS. 10a, 10b, and 10c show perspective views of the safety guard. This unit is designed to prevent the air-entrainment window 36, shown in FIG. 8a, from becoming occluded with any material (i.e., clothing). Said safety guard is also equipped with multiple safety guard slots 50, and a plurality of safety guard holes 52. Both the slots and holes are designed to allow for multiple routes of ambient air to reach said air-entrainment window 36 (FIG. 8a) in the event that any part of the safety guard becomes occluded. The final feature of the safety guard is a safety guard inlet hole 54 (FIG. 10c) which is present to allow said oxygen gas inlet 48 (FIG. 9a) to pass through and connect to said tubing adapter 56 (FIG. 11).

The next component in said air-entrainment system is shown in FIG. 8a. Its purpose is to set the desired percentage of oxygen to be delivered. Once said air-entrainment system is fully installed, an air-entrainment window 36 (FIG. 8a) is turned left or right to increase and decrease the oxygen percentage, respectfully. The other feature shown in FIG. 8a is a guide rim 38. Its presence allows a guide flange 46 (FIG. 9a) to sit inside said guide rim and allow for left and right movement without allowing the two from becoming disconnected.

The final component in said air-entrainment assembly is the shown in FIGS. 9a and 9b. This component has several features which should be noted. An oxygen gas inlet 48 is the first part of said air-entrainment assembly. It sends oxygen into an oxygen gas outlet 44. This outlet creates a stream of oxygen that decreases pressure inside FIGS. 9a and 9b, causing ambient air to enter through said air-entrainment window 36 (FIG. 8a) proportionally to the set flow, in accordance with venturi principles. Thus, the amount of oxygen flow can be adjusted without changing the oxygen percentage. Likewise, the flow of oxygen can remain constant, and the oxygen percentage can be adjusted by turning FIGS. 8a and 8b to a desired oxygen percentage. To set the oxygen percentage, FIGS. 9a and 9b have an oxygen percentage display 42 located on the current embodiment. Setting the desired oxygen percentage simply requires placing said air-entrainment window 36 below the percentage on the display. Also present in FIGS. 9a and 9b is securing unit 28 which is used to hold an air-entrainment outlet 40 to a reservoir inlet 34 in FIG. 7.

Said reservoir bag in FIG. 7, in this current embodiment, holds approximately five-hundred milliliters. This number was chosen based on the fact that an average sized breath for most individuals is five-hundred milliliters, thus giving the user a total breath that will meet his/her full inspiratory breath needs. The said reservoir bag leads the oxygen/oxygen mixture from the reservoir inlet 34 to the reservoir bag outlet 32 (FIG. 7). Attached to the bag outlet is the base of a one-way valve assembly which holds the two pieces in place by means of another securing unit 28 (FIGS. 5a, 5b, and 5c).

Said one-way valve assembly, shown in FIGS. 5a, 5b, and 5c, is needed to allow the oxygen to fill said reservoir bag while the user is exhaling, but opens when the users begins to inhale. This allows the system to conserve oxygen. A one-way valve 26 is of a flexible material, and it is supported by base supports 31 shown in FIG. 6b. Said one-way valve is held in place during inhalation by a retaining unit 30 shown in FIGS. 6a and 6b. Said one-way valve assembly attaches to a cap, shown in FIG. 4, where supply tube attachment 24 is located.

After placing the supply tubes 21 of the nasal cannula, shown in FIG. 2a, through the holes 22 in a retaining clip, shown in FIG. 3, the supply tubes 21 (FIG. 2a) are then pressed onto said supply tube attachment 24 (FIG. 4). Said retaining clip in FIG. 3 is designed to keep the current embodiment in place while in use by the user. The clip is slid up under the chin of the user to decrease the motion of said nasal prongs 20 (FIG. 2a).

After the current embodiment is assembled, it should be connected to a standard oxygen delivery device, the desired oxygen percentage should be set, and flow should be started. Once said reservoir bag (FIG. 7) is filled, it can be properly placed by inserting the nasal prongs 20 (FIG. 2b) in the nares of the user. Next, place a supply tube over and behind each ear. Slide the retaining clip up under the user's chin and adjust for comfort. Upon completion of this step, begin titrating the liter flow of the oxygen until said reservoir bag begins to partially deflate with each breath, but not fully deflating with any breath. Once achieved, this is the highest liter flow that the user requires at that time.

When the current embodiment is placed, as stated above, it will be able to: provide an efficient and precise amount of oxygen, up to one-hundred percent, while conserving said oxygen upon exhalation.

### CONCLUSION, RAMIFICATIONS, AND SCOPE

Thus the reader will see that at least one embodiment of the oxygen delivery system provides a more portable, lightweight, efficient, and accurate means for delivering a precise amount of oxygen. The current embodiment is able to meet the varying oxygen demands of its users in a variety of settings. The current embodiment may be more economical for the user, the payment source (Medicaid, Medicare, private insurers, etc.), and for the healthcare entity by allowing for the conservation of oxygen and shorter lengths of stay.

Whereby shortening the user's length of stay in a healthcare institution there is a resultant decreased risk of hospital acquired infections which increase the user's cost, morbidity, and/or mortality. Another advantage is early mobility for users with high oxygen demands. This mobility will cause the user to breathe deeper, which is common with activity, thus decreasing the risk for and helping to improve many pulmonary issues. In addition, early mobility lowers the risk for deep vein clots that can lead to pulmonary emboli (blood clots in the lungs) which alone increases oxygen demand and in some cases is fatal.

While the above description contains much specificity, this should not be construed as a limitation on the scope, but rather as an exemplification of one or more embodiments thereof. For example:
the air-entrainment system and reservoir bag may be relocated to allow for increased user comfort while maintaining efficiency;
the nasal prongs may have many differing sizes to allow for proper sealing of the user's nares;
the reservoir bag may be of differing sizes and material;
the cannula may be of differing diameters, lengths, and materials;
the nasal cannula may have a one-way flap at the base of the nasal prongs to allow for a user to exhale out of their nose without affecting the efficiency of the embodiment; and
the oxygen delivery system may utilize varying source gases in place of or in addition to oxygen such as: helium, nitrous oxide, nitric oxide, etc.

The oxygen delivery system has been described in its current embodiment, and it is clear that it is susceptible to numerous modifications, modes, and embodiments within the ability of those skilled in the art and without the exercise of the inventive faculty. Accordingly, the scope of the embodiments should be determined by the appended claims and their legal equivalents, rather than by the examples given.

1. A conserving, adjustable oxygen delivery system comprising: of an adjustable air-entrainment assembly, a reservoir bag, at least a single one-way valve, and nasal prongs;

2. The system as claimed in claim 1 wherein:
Said adjustable air-entrainment assembly utilizes venturi principles to allow for a controlled dilution of oxygen with ambient air,
whereby delivering an exact percentage of oxygen.

3. The system as claimed in claim 1 wherein:
Said adjustable air-entrainment assembly delivers an adjustable range of oxygen up to one-hundred percent,
whereby being able to meet a user's full oxygen demand.

4. The system as claimed in claim 1 wherein:
Said adjustable air-entrainment assembly allows for adjustments in oxygen percentage without having to change the flow of oxygen being delivered,
whereby allowing for a user's oxygen demand to be met independently of oxygen flow.

5. The system as claimed in claim 1 wherein:
Said adjustable air-entrainment assembly, utilizing venturi principles, allows the percentage of oxygen being delivered to remain the same when changes in flow occur,
whereby allowing for a user's inspiratory demand to be met independently of oxygen demand.

6. The system as claimed in claim 1 wherein:
Said reservoir bag stores the flow of oxygen during a user's exhalation, and is of sufficient volume to meet and average breath's need,
whereby conserving the flow of oxygen during exhalation, and storing enough oxygen to meet the next breath's need.

7. The system as claimed in claim 1 wherein:
Said one-way valve closes during a user's exhalation both allowing for said reservoir bag to inflate with the flow of oxygen, and to prevent exhaled gases from entering into the system,
whereby allowing for the conservation of the oxygen flow.

8. The system as claimed in claim 1 wherein:
Said nasal prongs are of sufficient size to create a seal in a user's nares that prevents ambient air entrainment into the user's nares during inspiration,
whereby preventing dilution of the percentage of oxygen being delivered.
